(19) United States
(12) Patent Application Publication
(10) Pub. No.: US 2018/0169326 A1
(43) Pub. Date: Jun. 21, 2018

(54) TAPERED FRONT-LOAD POWER INJECTOR SYRINGE

(71) Applicant: Liebel-Flarsheim Company LLC, Cincinnati, OH (US)

(72) Inventor: Frank M. Fago, Mason, OH (US)

(73) Assignee: Liebel-Flarsheim Company LLC, Cincinnati, OH (US)

(21) Appl. No.: 15/384,599

(22) Filed: Dec. 20, 2016

(51) Int. Cl. A61M 5/145; A61M 5/31; A61M 5/14

(52) U.S. Cl. CPC A61M 5/14546; A61M 5/1418; A61M 5/3129

(57) ABSTRACT

A power injector syringe assembly (100) that includes a syringe body (110) having a syringe barrel (120). An outer wall (126b) of the syringe barrel (120) is tapered from its proximal end (122) to its distal end (124), with the outer diameter of the syringe barrel (120) progressively increasing when proceeding in the direction of its distal end (124). When this syringe barrel (120) is disposed within a pressure sleeve (180), the clearance between the outer diameter of the syringe barrel (120) and the inner diameter of the pressure sleeve (180) is progressively reduced proceeding in the discharge direction during power injector operations.

### FIELD OF THE INVENTION

The present invention generally relates to the field of power injectors and, more particularly, to syringes for use with power injectors.

### BACKGROUND

Various medical procedures require that one or more medical fluids be injected into a patient. For example, medical imaging procedures oftentimes involve the injection of contrast media into a patient, possibly along with saline and/or other fluids. Other medical procedures involve injecting one or more fluids into a patient for therapeutic purposes. Power injectors may be used for these types of applications.

A power injector generally includes what is commonly referred to as a powerhead. One or more syringes may be mounted to the powerhead in various manners (e.g., detachably; rear-loading; front-loading; side-loading). Each syringe typically includes what may be characterized as a syringe plunger, piston, or the like. Each such syringe plunger is designed to interface with (e.g., contact and/or temporarily interconnect with) an appropriate syringe plunger driver that is incorporated into the powerhead, such that operation of the syringe plunger driver axially advances the associated syringe plunger inside and relative to a barrel of the syringe. One typical syringe plunger driver is in the form of a ram that is mounted on a threaded lead or drive screw. Rotation of the drive screw in one rotational direction advances the associated ram in one axial direction, while rotation of the drive screw in the opposite rotational direction advances the associated ram in the opposite axial direction.

In at least some instances, a pressure jacket or sleeve is used in conjunction with a power injector syringe. The pressure jacket is installed on the power injector, and the power injector syringe is disposed inside the pressure jacket for execution of an injection procedure. Generally, the pressure jacket is configured to have a higher pressure resistance than the power injector syringe. As such, the pressure jacket is typically used for multiple injection procedures, and the power injector syringe is configured as a disposable (e.g., used for single injection procedure). The pressure jacket function is to limit the amount that the power injector syringe can expand during an injection, and to provide sufficient structural integrity for the pressures that may be experienced during a given injection procedure.

Reducing the amount of clearance between the outer diameter of the syringe and the inner diameter of the pressure sleeve should reduce the amount that the syringe may expand before being restrained (and thereby radially supported) by the pressure sleeve, and which should also reduce the potential of losing a seal between the plunger of the syringe and the syringe body. However, such a reduced clearance may also adversely impact the installation of the syringe within the pressure sleeve.

### SUMMARY

A first aspect of the present invention is directed to a power injector syringe assembly that includes a syringe body and a piston, where the syringe body includes a syringe barrel having an inner diameter and an outer diameter that are each disposed about a central axis of the syringe body. The outer diameter of the syringe barrel continually increases proceeding from a first location to a second location, where these first and second locations may be characterized as being spaced along/relative to the central axis. Both a discharge orifice for the syringe body and the second location are spaced from the first location in a common direction.

A number of feature refinements and additional features are applicable to the first aspect of the present invention. These feature refinements and additional features may be used individually or in any combination. As such, each of the following features that will be discussed may be, but are not required to be, used with any other feature or combination of features of the first aspect. One advantage of the first aspect is facilitating installation of the syringe within a pressure sleeve.

The syringe barrel may include an outer wall and an inner wall. The inner wall of the syringe barrel may be cylindrical, while the outer wall of the syringe barrel may include a taper that extends from the noted first location to the second location. The noted taper may be characterized as existing for the entire length of the syringe barrel (e.g., the entirety of the outer wall for the syringe barrel may be tapered proceeding from the first location to the second location). As such, an open end of the syringe barrel may coincide with the first location.

The tapering of at least part of the outer wall for the syringe barrel may be of a constant or fixed magnitude. One embodiment has the magnitude of this taper being constant and no more than about 10°. Another embodiment has the magnitude of this taper being constant and within a range of about 0.5° to about 10°. Yet another embodiment has the magnitude of this taper being constant and about 5°. Such a taper may be measured relative to an axis that is parallel to the central axis for the syringe body.

The syringe barrel may be characterized as including first and second ends that are opposite of one another and that are spaced along the central axis for the syringe body. The first end of the syringe barrel may be referred to as a proximal end (e.g., an open end of the syringe barrel that projects toward a power injector when the power injector syringe assembly is in an installed configuration), while the second end of the syringe barrel may be referred to as a distal or remote end (e.g., spaced furthest from the power injector when the power injector syringe assembly is in an installed configuration). The syringe body may further include a frustumly-shaped section that extends from the second or distal end of the syringe body, and that converges toward the central axis of the syringe body in proceeding away from the syringe barrel (e.g., the piston for the power injector syringe assembly may be advanced at least generally in the direction of the frustumly-shaped section during a power injector fluid discharge operation).

The noted first or proximal end of the syringe body may coincide with the first location, while the second or distal end of the syringe body may coincide with the second location. As such and as previously noted, the entire length of the syringe barrel (e.g., measured along/relative to the central axis), may correspond with a spacing or distance between the first and second locations (between which the outer diameter of the syringe barrel continually increases proceeding from the first location to the second location). The outer diameter of the syringe barrel may be characterized as continuously diverging relative to the central axis in proceeding from the first end of the syringe barrel to the second end of the syringe barrel. In contrast, the inner diameter of the syringe barrel may be of a constant magnitude in proceeding from the first end of the syringe barrel to the second end of the syringe barrel (e.g., a cylindrical configuration).

The syringe body may be characterized as including a discharge end section that in turn includes the noted discharge orifice. Such a discharge end section of the syringe body may include a discharge nozzle that in turn incorporates the noted discharge orifice. This discharge nozzle may include an appropriate connector (e.g., a Luer connector) for detachably connecting the syringe body with a tube that may be incorporated by the power injector syringe assembly. The piston may be advanced within the syringe barrel at least generally in the direction of the discharge nozzle during a power injector fluid discharge operation. This discharge nozzle could extend from the above-noted frustumly-shaped section, or more generally from an end wall for the syringe barrel that may be of any appropriate configuration.

The power injector syringe assembly may include one or more tubing clips, including a first tubing clip. Any appropriate number of tubing clips may be utilized, and multiple tubing clips may be disposed in any appropriate arrangement relative to one another. The first tubing clip includes a first tube receptacle (e.g., in the form of a concave opening) and that is accessible on an exterior of the power injector syringe assembly. A portion of a tube that is fluidly connected with the syringe body may be detachably received in the first tube receptacle for the first tubing clip.

One or more tubing clips may be incorporated at any appropriate location of the power injector syringe assembly. An end of the syringe body that incorporates the discharge orifice may be referred to as a “closed end” of the syringe body (the piston being moved toward the closed end of the syringe body for a fluid discharge operation). One or more tubing clips may be disposed on an exterior surface of such a closed end of the syringe body (including where such a closed end includes the above-noted frustumly-shaped section—one or more tubing clips could extend from an exterior of the above-noted frustumly-shaped section). A shield or cap may be disposed over the above-noted frustumly-shaped section of the syringe body, and including where such a shield is integrally formed with the syringe body or where such a shield is separately formed from and is appropriately secured relative to the syringe body. One or more of the noted tubing clips could be positioned on an exterior surface of such a shield.

The power injector syringe assembly may further include a pressure sleeve, with the syringe barrel being disposed within this pressure sleeve. The pressure sleeve may include an inner wall and an outer wall. The inner wall and the outer wall may be cylindrical surfaces, although other configurations may be appropriate. A constant wall thickness may be utilized along the entire length of the pressure sleeve, although such may not be required in all instances. The maximum spacing between the outer wall of the syringe barrel and the inner wall of the pressure sleeve coincides with the first location, while the minimum spacing between the outer wall of the syringe barrel and the inner wall of the pressure sleeve coincides with the second location. The spacing between the outer wall of the syringe barrel and the inner wall of the pressure sleeve may be reduced at a constant rate proceeding from the first location to the second location. The outer wall of the syringe barrel and the inner wall of the pressure sleeve may be spaced from one another at the second location, or the outer wall the syringe barrel could actually be in contact with the inner wall of the pressure sleeve at the second location (all prior to movement of the piston within the syringe barrel pursuant to a power injector fluid discharge operation).

The power injector assembly may include one or more of the noted tubing clips, along with the noted pressure sleeve. In this regarding, each such tubing clip will be disposed outside of the pressure sleeve. Consider the case where the power injector syringe assembly includes the above-noted shield or cap. This shield would be disposed beyond the pressure sleeve when the syringe barrel is disposed within the pressure sleeve. An outer perimeter of this shield may be positioned radially beyond the outer perimeter of the pressure sleeve (e.g., spaced further from the central axis of the syringe body) for purposes of reducing the potential for fluids entering any space between the pressure sleeve and the syringe barrel. One or more of the noted tubing clips could be positioned on an exterior surface of such a shield. The above-noted frustumly-shaped section of the syringe body could extend beyond the pressure sleeve, could define an exterior of the power injector syringe assembly, and could include one or more of the noted tubing clips. In this instance, the syringe body could include an annular flange that protrudes beyond the outer perimeter of the pressure sleeve in proximity to an adjacent end of the pressure sleeve for purposes of reducing the potential for fluids entering any space between the pressure sleeve and the syringe barrel.

A second aspect of the present invention is directed to a power injector syringe assembly having a syringe body with a discharge orifice, along with a piston that is movably disposed within this syringe body. Movement of the piston in the direction of the discharge orifice, pursuant to a power injector fluid discharge operation, will discharge fluid out of the syringe body through the discharge orifice. The power injector syringe assembly further includes at least one tubing clip having a tube receptacle that is accessible on an exterior of the power injector syringe assembly.

A number of feature refinements and additional features are applicable to the second aspect of the present invention. These feature refinements and additional features may be used individually or in any combination. As such, each of the following features that will be discussed may be, but are not required to be, used with any other feature or combination of features of the second aspect.

The syringe body may include both a syringe barrel and an end section, where the end section incorporates the discharge orifice. The syringe barrel may extend from an open end (e.g., that would face in the direction of a powerhead with the power injector syringe assembly being in an installed configuration) to this end section (and which may be referred to as a closed end for the syringe body and that includes the noted discharge orifice). One or more tubing clips may be incorporated by this end section. One embodiment has the end section including a shield that is positioned over a frustumly-shaped section, where this frustumly-shaped section proceeds from the syringe barrel in converging relation to a central axis for the syringe body, and where one or more tubing clips are incorporated on an exterior of this shield. Another embodiment has this end section including a frustumly-shaped section that proceeds from the syringe barrel in converging relation to a central axis for the syringe body, and where one or more tubing clips are incorporated on an exterior of this frustumly-shaped section. Yet another embodiment has this end section including an end wall that is at least generally orthogonal to a flow through the syringe body, where one or more tubing clips are incorporated on an exterior surface of this end wall.

Fluid may be contained within the syringe body between the piston and the discharge orifice. Advancement of the piston relative to the syringe body and in the direction of the discharge orifice (e.g., by a power injector fluid discharge operation) will then discharge fluid from the syringe body through this discharge orifice. A tube may be fluidly connected with the discharge orifice to accept a flow out of the syringe body, including where this tube is detachably connected with the syringe body (e.g., a discharge nozzle or tip that includes the discharge orifice). A portion of this tube may be detachably positioned within the tube receptacle of one or more tubing clips that are utilized by the power injector syringe assembly.

Any power injector that may be utilized by the present invention to provide a fluid discharge may be of any appropriate size, shape, configuration, and/or type. Any such power injector may utilize one or more syringe plunger drivers of any appropriate size, shape, configuration, and/or type, where each such syringe plunger driver is capable of bi-directional movement (e.g., a movement in a first direction for discharging fluid; a movement in a second direction for accommodating a loading and/or drawing of fluid and/or so as to return to a position for a subsequent fluid discharge operation), and where each such syringe plunger driver may interact with its corresponding syringe plunger in any appropriate manner (e.g., by mechanical contact; by an appropriate coupling (mechanical or otherwise)) so as to be able to advance the syringe plunger in at least one direction (e.g., to discharge fluid). Each syringe plunger driver may utilize one or more drive sources of any appropriate size, shape, configuration, and/or type. Multiple drive source outputs may be combined in any appropriate manner to advance a single syringe plunger at a given time. One or more drive sources may be dedicated to a single syringe plunger driver, one or more drive sources may be associated with multiple syringe plunger drivers (e.g., incorporating a transmission of sorts to change the output from one syringe plunger to another syringe plunger), or a combination thereof. Representative drive source forms include a brushed or brushless electric motor, a hydraulic motor, a pneumatic motor, a piezoelectric motor, or a stepper motor.

Any such power injector may be used for any appropriate application where the delivery of one or more medical fluids is desired, including without limitation any appropriate medical imaging application (e.g., computed tomography or CT imaging; magnetic resonance imaging or MRI; single photon emission computed tomography or SPECT imaging; positron emission tomography or PET imaging; X-ray imaging; angiographic imaging; optical imaging; ultrasound imaging) and/or any appropriate medical diagnostic and/or therapeutic application (e.g., injection of chemotherapy, pain management, etc.). Any such power injector may be used in conjunction with any component or combination of components, such as an appropriate imaging system (e.g., a CT scanner). For instance, information could be conveyed between any such power injector and one or more other components (e.g., scan delay information, injection start signal, injection rate).

Any appropriate number of syringes may be utilized with any such power injector in any appropriate manner (e.g., detachably; front-loaded; rear-loaded; side-loaded), any appropriate medical fluid may be discharged from a given syringe of any such power injector (e.g., contrast media, therapeutic fluid, a radiopharmaceutical, saline, and any combination thereof), and any appropriate fluid may be discharged from a multiple syringe power injector configuration in any appropriate manner (e.g., sequentially, simultaneously), or any combination thereof. In one embodiment, fluid discharged from a syringe by operation of the power injector is directed into a conduit (e.g., medical tubing set), where this conduit is fluidly interconnected with the syringe in any appropriate manner and directs fluid to a desired location (e.g., to a catheter that is inserted into a patient for injection). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). In one embodiment, each syringe includes a syringe barrel and a plunger that is disposed within and movable relative to the syringe barrel. This plunger may interface with the power injector's syringe plunger drive assembly such that the syringe plunger drive assembly is able to advance the plunger in at least one direction, and possibly in two different, opposite directions.

### BRIEF DESCRIPTION OF THE FIGURES

FIG. 1 is a schematic of one embodiment of a power injector.

FIG. 2A is a perspective view of one embodiment of a portable stand-mounted, dual-head power injector.

FIG. 2B is an enlarged, partially exploded, perspective view of a powerhead used by the power injector of FIG. 2A.

FIG. 2C is a schematic of one embodiment of a syringe plunger drive assembly used by the power injector of FIG. 2A.

FIG. 3A is a perspective view of one embodiment of a power injector syringe assembly having a tapered syringe barrel.

FIG. 3B is an enlarged perspective view of the power injector syringe assembly of FIG. 3A, and that illustrates an associated extension tube.

FIG. 4 is a cross-sectional view of the power injector syringe assembly of FIG. 3A, with a piston being shown in a retracted position.

FIG. 5 is another cross-sectional view of the power injector syringe assembly of FIG. 3A, and with the piston being shown in an extended position.

FIG. 6 is a perspective view of one embodiment of a pressure sleeve that may be incorporated by the power injector syringe assembly of FIG. 3A.

FIG. 7 is an enlarged, cross-sectional schematic of the power injector syringe assembly of FIG. 3A for the case of including a pressure sleeve, and that illustrates the taper of the outer wall of the syringe barrel.

### DETAILED DESCRIPTION

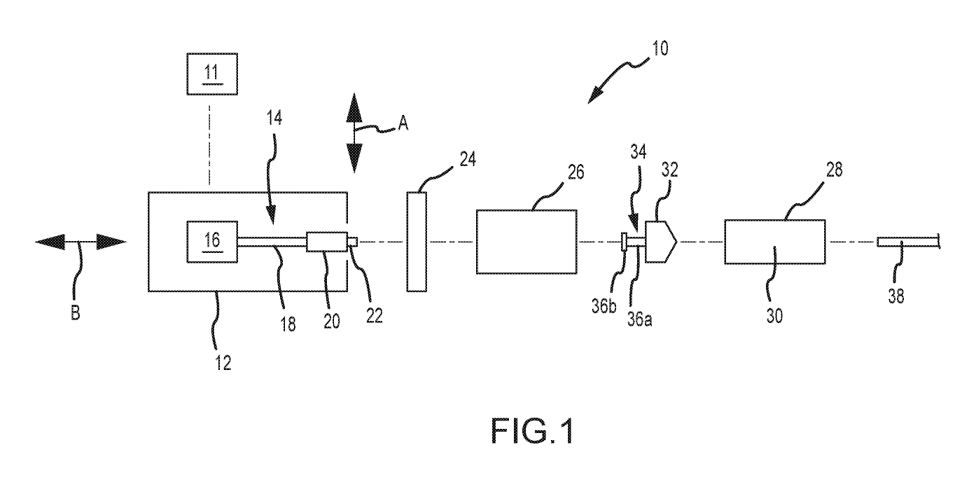

FIG. 1 presents a schematic of one embodiment of a power injector 10 having a powerhead 12. One or more graphical user interfaces or GUIs 11 may be associated with the powerhead 12. Each GUI 11: 1) may be of any appropriate size, shape, configuration, and/or type; 2) may be operatively interconnected with the powerhead 12 in any appropriate manner; 3) may be disposed at any appropriate location; 4) may be configured to provide any of the following functions: controlling one or more aspects of the operation of the power injector 10; inputting/editing one or more parameters associated with the operation of the power injector 10; and displaying appropriate information (e.g., associated with the operation of the power injector 10); or 5) any combination of the foregoing. Any appropriate number of GUIs 11 may be utilized. In one embodiment, the power injector 10 includes a GUI 11 that is incorporated by a console that is separate from but which communicates with the powerhead 12. In another embodiment, the power injector 10 includes a GUI 11 that is part of the powerhead 12. In yet another embodiment, the power injector 10 utilizes one GUI 11 on a separate console that communicates with the powerhead 12, and also utilizes another GUI 11 that is on the powerhead 12. Each GUI 11 could provide the same functionality or set of functionalities, or the GUIs 11 may differ in at least some respect in relation to their respective functionalities.

A syringe 28 may be installed on the powerhead 12 and, when installed, may be considered to be part of the power injector 10. Some injection procedures may result in a relatively high pressure being generated within the syringe 28. In this regard, it may be desirable to dispose the syringe 28 within a pressure jacket 26. The pressure jacket 26 is typically associated with the powerhead 12 in a manner that allows the syringe 28 to be disposed therein as a part of or after installing the syringe 28 on the powerhead 12. The same pressure jacket 26 will typically remain associated with the powerhead 12, as various syringes 28 are positioned within and removed from the pressure jacket 26 for multiple injection procedures. The power injector 10 may eliminate the pressure jacket 26 if the power injector 10 is configured/utilized for low-pressure injections and/or if the syringe(s) 28 to be utilized with the power injector 10 is (are) of sufficient durability to withstand high-pressure injections without the additional support provided by a pressure jacket 26. In any case, fluid discharged from the syringe 28 may be directed into a conduit 38 of any appropriate size, shape, configuration, and/or type, which may be fluidly interconnected with the syringe 28 in any appropriate manner, and which may direct fluid to any appropriate location (e.g., to a patient).

The powerhead 12 includes a syringe plunger drive assembly or syringe plunger driver 14 that interacts (e.g., interfaces) with the syringe 28 (e.g., a plunger 32 thereof) to discharge fluid from the syringe 28. This syringe plunger drive assembly 14 includes a drive source 16 (e.g., a motor of any appropriate size, shape, configuration, and/or type, optional gearing, and the like) that powers a drive output 18 (e.g., a rotatable drive screw). A ram 20 may be advanced along an appropriate path (e.g., axial) by the drive output 18. The ram 20 may include a coupler 22 for interacting or interfacing with a corresponding portion of the syringe 28 in a manner that will be discussed below.

The syringe 28 includes a plunger or piston 32 that is movably disposed within a syringe barrel 30 (e.g., for axial reciprocation along an axis coinciding with the double-headed arrow B). The plunger 32 may include a coupler 34. This syringe plunger coupler 34 may interact or interface with the ram coupler 22 to allow the syringe plunger drive assembly 14 to retract the syringe plunger 32 within the syringe barrel 30. The syringe plunger coupler 34 may be in the form of a shaft 36a that extends from a body of the syringe plunger 32, together with a head or button 36b. However, the syringe plunger coupler 34 may be of any appropriate size, shape, configuration, and/or type.

Generally, the syringe plunger drive assembly 14 of the power injector 10 may interact with the syringe plunger 32 of the syringe 28 in any appropriate manner (e.g., by mechanical contact; by an appropriate coupling (mechanical or otherwise)) so as to be able to move or advance the syringe plunger 32 (relative to the syringe barrel 30) in at least one direction (e.g., to discharge fluid from the corresponding syringe 28). That is, although the syringe plunger drive assembly 14 may be capable of bi-directional motion (e.g., via operation of the same drive source 16), the power injector 10 may be configured such that the operation of the syringe plunger drive assembly 14 actually only moves each syringe plunger 32 being used by the power injector 10 in only one direction. However, the syringe plunger drive assembly 14 may be configured to interact with each syringe plunger 32 being used by the power injector 10 so as to be able to move each such syringe plunger 32 in each of two different directions (e.g. in different directions along a common axial path).

Retraction of the syringe plunger 32 may be utilized to accommodate a loading of fluid into the syringe barrel 30 for a subsequent injection or discharge, may be utilized to actually draw fluid into the syringe barrel 30 for a subsequent injection or discharge, or for any other appropriate purpose. Certain configurations may not require that the syringe plunger drive assembly 14 be able to retract the syringe plunger 32, in which case the ram coupler 22 and syringe plunger coupler 34 may not be desired. In this case, the syringe plunger drive assembly 14 may be retracted for purposes of executing another fluid delivery operation (e.g., after another pre-filled syringe 28 has been installed). Even when a ram coupler 22 and syringe plunger coupler 34 are utilized, these components may or may not be coupled when the ram 20 advances the syringe plunger 32 to discharge fluid from the syringe 28 (e.g., the ram 20 may simply “push on” the syringe plunger coupler 34 or directly on a proximal end of the syringe plunger 32). Any single motion or combination of motions in any appropriate dimension or combination of dimensions may be utilized to dispose the ram coupler 22 and syringe plunger coupler 34 in a coupled state or condition, to dispose the ram coupler 22 and syringe plunger coupler 34 in an un-coupled state or condition, or both.

The syringe 28 may be installed on the powerhead 12 in any appropriate manner. For instance, the syringe 28 could be configured to be installed directly on the powerhead 12. In the illustrated embodiment, a housing 24 is appropriately mounted on the powerhead 12 to provide an interface between the syringe 28 and the powerhead 12. This housing 24 may be in the form of an adapter to which one or more configurations of syringes 28 may be installed, and where at least one configuration for a syringe 28 could be installed directly on the powerhead 12 without using any such adapter. The housing 24 may also be in the form of a faceplate to which one or more configurations of syringes 28 may be installed. In this case, it may be such that a faceplate is required to install a syringe 28 on the powerhead 12—the syringe 28 could not be installed on the powerhead 12 without the faceplate. When a pressure jacket 26 is being used, it may be installed on the powerhead 12 in the various manners discussed herein in relation to the syringe 28, and the syringe 28 will then thereafter be installed in the pressure jacket 26.

The housing 24 may be mounted on and remain in a fixed position relative to the powerhead 12 when installing a syringe 28. Another option is to movably interconnect the housing 24 and the powerhead 12 to accommodate installing a syringe 28. For instance, the housing 24 may move within a plane that contains the double-headed arrow A to provide one or more of coupled state or condition and an un-coupled state or condition between the ram coupler 22 and the syringe plunger coupler 34.

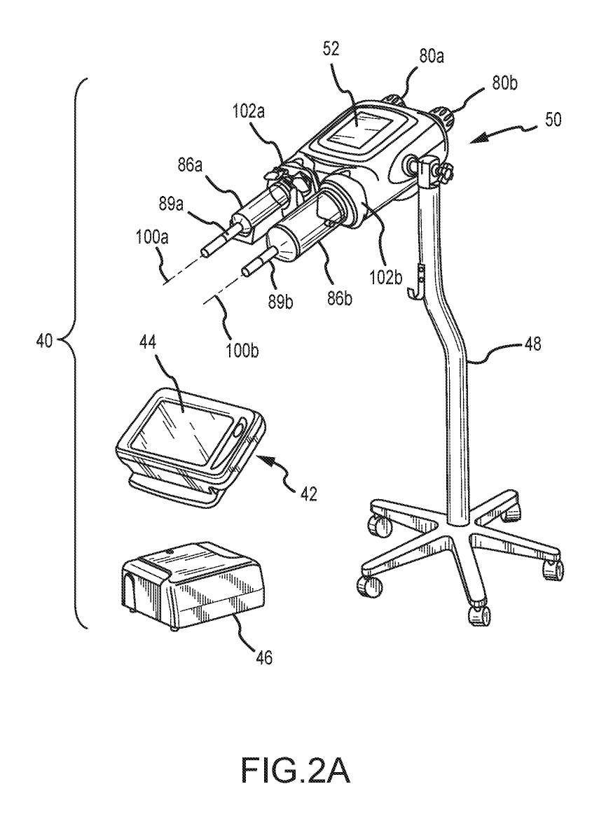

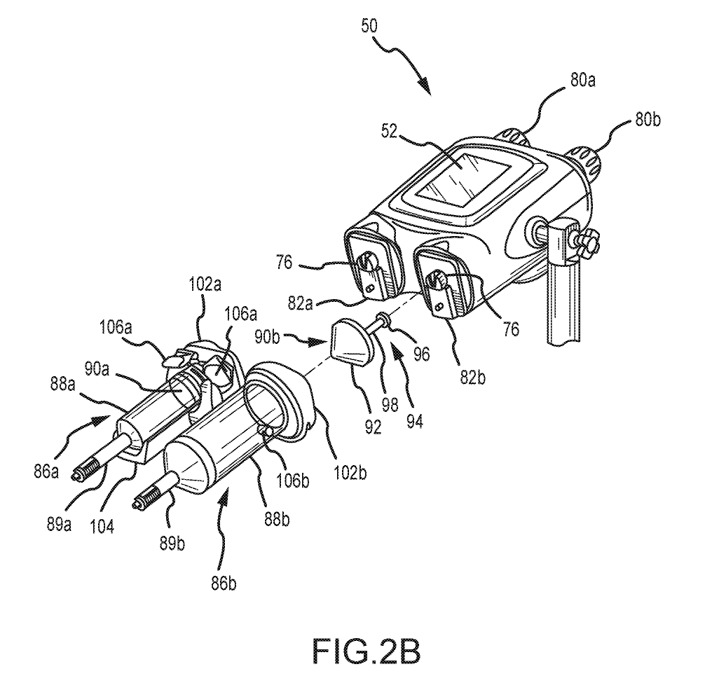

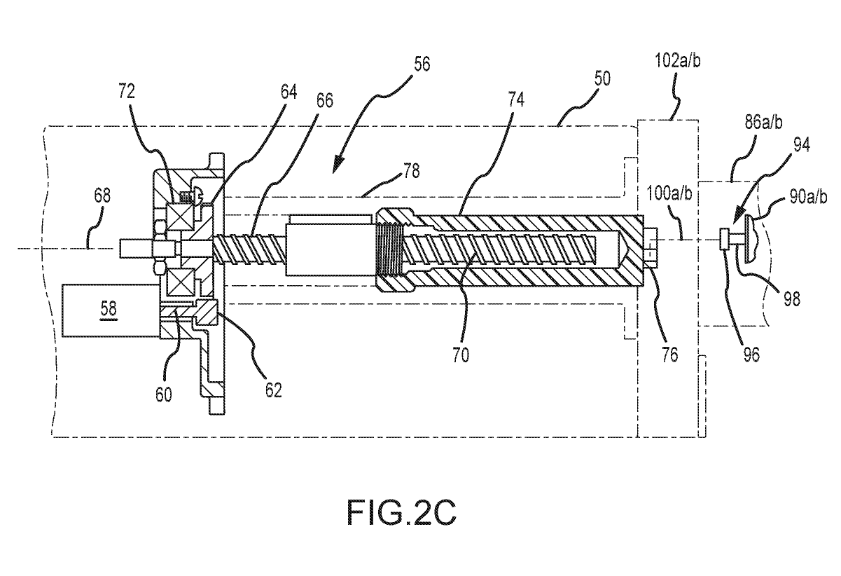

One particular power injector configuration is illustrated in FIG. 2A, is identified by a reference numeral 40, and is at least generally in accordance with the power injector 10 of FIG. 1. The power injector 40 includes a powerhead 50 that is mounted on a portable stand 48. Two syringes 86a, 86b for the power injector 40 are mounted on the powerhead 50. Fluid may be discharged from the syringes 86a, 86b during operation of the power injector 40.

The portable stand 48 may be of any appropriate size, shape, configuration, and/or type. Wheels, rollers, casters, or the like may be utilized to make the stand 48 portable. The powerhead 50 could be maintained in a fixed position relative to the portable stand 48. However, it may be desirable to allow the position of the powerhead 50 to be adjustable relative to the portable stand 48 in at least some manner. For instance, it may be desirable to have the powerhead 50 in one position relative to the portable stand 48 when loading fluid into one or more of the syringes 86a, 86b, and to have the powerhead 50 in a different position relative to the portable stand 48 for performance of an injection procedure. In this regard, the powerhead 50 may be movably interconnected with the portable stand 48 in any appropriate manner (e.g., such that the powerhead 50 may be pivoted through at least a certain range of motion, and thereafter maintained in the desired position).

It should be appreciated that the powerhead 50 could be supported in any appropriate manner for providing fluid. For instance, instead of being mounted on a portable structure, the powerhead 50 could be interconnected with a support assembly, that in turn is mounted to an appropriate structure (e.g., ceiling, wall, floor). Any support assembly for the powerhead 50 may be positionally adjustable in at least some respect (e.g., by having one or more support sections that may be repositioned relative to one or more other support sections), or may be maintained in a fixed position. Moreover, the powerhead 50 may be integrated with any such support assembly so as to either be maintained in a fixed position or so as to be adjustable relative the support assembly.

The powerhead 50 includes a graphical user interface or GUI 52. This GUI 52 may be configured to provide one or any combination of the following functions: controlling one or more aspects of the operation of the power injector 40; inputting/editing one or more parameters associated with the operation of the power injector 40; and displaying appropriate information (e.g., associated with the operation of the power injector 40). The power injector 40 may also include a console 42 and powerpack 46 that each may be in communication with the powerhead 50 in any appropriate manner (e.g., via one or more cables), that may be placed on a table or mounted on an electronics rack in an examination room or at any other appropriate location, or both. The powerpack 46 may include one or more of the following and in any appropriate combination: a power supply for the injector 40; interface circuitry for providing communication between the console 42 and powerhead 50; circuitry for permitting connection of the power injector 40 to remote units such as remote consoles, remote hand or foot control switches, or other original equipment manufacturer (OEM) remote control connections (e.g., to allow for the operation of power injector 40 to be synchronized with the x-ray exposure of an imaging system); and any other appropriate componentry. The console 42 may include a touch screen display 44, which in turn may provide one or more of the following functions and in any appropriate combination: allowing an operator to remotely control one or more aspects of the operation of the power injector 40; allowing an operator to enter/edit one or more parameters associated with the operation of the power injector 40; allowing an operator to specify and store programs for automated operation of the power injector 40 (which can later be automatically executed by the power injector 40 upon initiation by the operator); and displaying any appropriate information relation to the power injector 40 and including any aspect of its operation.

Various details regarding the integration of the syringes 86a, 86b with the powerhead 50 are presented in FIG. 2B. Each of the syringes 86a, 86b includes the same general components. The syringe 86a includes plunger or piston 90a that is movably disposed within a syringe barrel 88a. Movement of the plunger 90a along an axis 100a (FIG. 2A) via operation of the powerhead 50 will discharge fluid from within a syringe barrel 88a through a nozzle 89a of the syringe 86a. An appropriate conduit (not shown) will typically be fluidly interconnected with the nozzle 89a in any appropriate manner to direct fluid to a desired location (e.g., a patient). Similarly, the syringe 86b includes plunger or piston 90b that is movably disposed within a syringe barrel 88b. Movement of the plunger 90b along an axis 100b (FIG. 2A) via operation of the powerhead 50 will discharge fluid from within the syringe barrel 88b through a nozzle 89b of the syringe 86b. An appropriate conduit (not shown) will typically be fluidly interconnected with the nozzle 89b in any appropriate manner to direct fluid to a desired location (e.g., a patient).

The syringe 86a is interconnected with the powerhead 50 via an intermediate faceplate 102a. This faceplate 102a includes a cradle 104 that supports at least part of the syringe barrel 88a, and which may provide/accommodate any additional functionality or combination of functionalities. A mounting 82a is disposed on and is fixed relative to the powerhead 50 for interfacing with the faceplate 102a. A ram coupler 76 of a ram 74 (FIG. 2C), which are each part of a syringe plunger drive assembly or syringe plunger driver 56 (FIG. 2C) for the syringe 86a, is positioned in proximity to the faceplate 102a when mounted on the powerhead 50. Details regarding the syringe plunger drive assembly 56 will be discussed in more detail below in relation to FIG. 2C. Generally, the ram coupler 76 may be coupled with the syringe plunger 90a of the syringe 86a, and the ram coupler 76 and ram 74 (FIG. 2C) may then be moved relative to the powerhead 50 to move the syringe plunger 90a along the axis 100a (FIG. 2A). It may be such that the ram coupler 76 is engaged with, but not actually coupled to, the syringe plunger 90a when moving the syringe plunger 90a to discharge fluid through the nozzle 89a of the syringe 86a.

The faceplate 102a may be moved at least generally within a plane that is orthogonal to the axes 100a, 100b (associated with movement of the syringe plungers 90a, 90b, respectively, and illustrated in FIG. 2A), both to mount the faceplate 102a on and remove the faceplate 102a from its mounting 82a on the powerhead 50. The faceplate 102a may be used to couple the syringe plunger 90a with its corresponding ram coupler 76 on the powerhead 50. In this regard, the faceplate 102a includes a pair of handles 106a. Generally and with the syringe 86a being initially positioned within the faceplate 102a, the handles 106a may be moved to in turn move/translate the syringe 86a at least generally within a plane that is orthogonal to the axes 100a, 100b (associated with movement of the syringe plungers 90a, 90b, respectively, and illustrated in FIG. 2A). Moving the handles 106a to one position moves/translates the syringe 86a (relative to the faceplate 102a) in an at least generally downward direction to couple its syringe plunger 90a with its corresponding ram coupler 76. Moving the handles 106a to another position moves/translates the syringe 86a (relative to the faceplate 102a) in an at least generally upward direction to uncouple its syringe plunger 90a from its corresponding ram coupler 76.

The syringe 86b is interconnected with the powerhead 50 via an intermediate faceplate 102b. A mounting 82b is disposed on and is fixed relative to the powerhead 50 for interfacing with the faceplate 102b. A ram coupler 76 of a ram 74 (FIG. 2C), which are each part of a syringe plunger drive assembly 56 for the syringe 86b, is positioned in proximity to the faceplate 102b when mounted to the powerhead 50. Details regarding the syringe plunger drive assembly 56 again will be discussed in more detail below in relation to FIG. 2C. Generally, the ram coupler 76 may be coupled with the syringe plunger 90b of the syringe 86b, and the ram coupler 76 and ram 74 (FIG. 2C) may be moved relative to the powerhead 50 to move the syringe plunger 90b along the axis 100b (FIG. 2A). It may be such that the ram coupler 76 is engaged with, but not actually coupled to, the syringe plunger 90b when moving the syringe plunger 90b to discharge fluid through the nozzle 89b of the syringe 86b.

The faceplate 102b may be moved at least generally within a plane that is orthogonal to the axes 100a, 100b (associated with movement of the syringe plungers 90a, 90b, respectively, and illustrated in FIG. 2A), both to mount the faceplate 102b on and remove the faceplate 102b from its mounting 82b on the powerhead 50. The faceplate 102b also may be used to couple the syringe plunger 90b with its corresponding ram coupler 76 on the powerhead 50. In this regard, the faceplate 102b may include a handle 106b. Generally and with the syringe 86b being initially positioned within the faceplate 102b, the syringe 86b may be rotated along its long axis 100b (FIG. 2A) and relative to the faceplate 102b. This rotation may be realized by moving the handle 106b, by grasping and turning the syringe 86b, or both. In any case, this rotation moves/translates both the syringe 86b and the faceplate 102b at least generally within a plane that is orthogonal to the axes 100a, 100b (associated with movement of the syringe plungers 90a, 90b, respectively, and illustrated in FIG. 2A). Rotating the syringe 86b in one direction moves/translates the syringe 86b and faceplate 102b in an at least generally downward direction to couple the syringe plunger 90b with its corresponding ram coupler 76. Rotating the syringe 86b in the opposite direction moves/translates the syringe 86b and faceplate 102b in an at least generally upward direction to uncouple its syringe plunger 90b from its corresponding ram coupler 76.

As illustrated in FIG. 2B, the syringe plunger 90b includes a plunger body 92 and a syringe plunger coupler 94. This syringe plunger coupler 94 includes a shaft 98 that extends from the plunger body 92, along with a head 96 that is spaced from the plunger body 92. Each of the ram couplers 76 includes a larger slot that is positioned behind a smaller slot on the face of the ram coupler 76. The head 96 of the syringe plunger coupler 94 may be positioned within the larger slot of the ram coupler 76, and the shaft 98 of the syringe plunger coupler 94 may extend through the smaller slot on the face of the ram coupler 76 when the syringe plunger 90b and its corresponding ram coupler 76 are in a coupled state or condition. The syringe plunger 90a may include a similar syringe plunger coupler 94 for interfacing with its corresponding ram coupler 76.

The powerhead 50 is utilized to discharge fluid from the syringes 86a, 86b in the case of the power injector 40. That is, the powerhead 50 provides the motive force to discharge fluid from each of the syringes 86a, 86b. One embodiment of what may be characterized as a syringe plunger drive assembly or syringe plunger driver is illustrated in FIG. 2C, is identified by reference numeral 56, and may be utilized by the powerhead 50 to discharge fluid from each of the syringes 86a, 86b. A separate syringe plunger drive assembly 56 may be incorporated into the powerhead 50 for each of the syringes 86a, 86b. In this regard and referring back to FIGS. 2A-B, the powerhead 50 may include hand-operated knobs 80a and 80b for use in separately controlling each of the syringe plunger drive assemblies 56.

Initially and in relation to the syringe plunger drive assembly 56 of FIG. 2C, each of its individual components may be of any appropriate size, shape, configuration and/or type. The syringe plunger drive assembly 56 includes a motor 58, which has an output shaft 60. A drive gear 62 is mounted on and rotates with the output shaft 60 of the motor 58. The drive gear 62 is engaged or is at least engageable with a driven gear 64. This driven gear 64 is mounted on and rotates with a drive screw or shaft 66. The axis about which the drive screw 66 rotates is identified by reference numeral 68. One or more bearings 72 appropriately support the drive screw 66.

A carriage or ram 74 is movably mounted on the drive screw 66. Generally, rotation of the drive screw 66 in one direction axially advances the ram 74 along the drive screw 66 (and thereby along axis 68) in the direction of the corresponding syringe 86a/b, while rotation of the drive screw 66 in the opposite direction axially advances the ram 74 along the drive screw 66 (and thereby along axis 68) away from the corresponding syringe 86a/b. In this regard, the perimeter of at least part of the drive screw 66 includes helical threads 70 that interface with at least part of the ram 74. The ram 74 is also movably mounted within an appropriate bushing 78 that does not allow the ram 74 to rotate during a rotation of the drive screw 66. Therefore, the rotation of the drive screw 66 provides for an axial movement of the ram 74 in a direction determined by the rotational direction of the drive screw 66.

The ram 74 includes a coupler 76 that that may be detachably coupled with a syringe plunger coupler 94 of the syringe plunger 90a/b of the corresponding syringe 86a/b. When the ram coupler 76 and syringe plunger coupler 94 are appropriately coupled, the syringe plunger 90a/b moves along with ram 74. FIG. 2C illustrates a configuration where the syringe 86a/b may be moved along its corresponding axis 100a/b without being coupled to the ram 74. When the syringe 86a/b is moved along its corresponding axis 100a/b such that the head 96 of its syringe plunger 90a/b is aligned with the ram coupler 76, but with the axes 68 still in the offset configuration of FIG. 2C, the syringe 86a/b may be translated within a plane that is orthogonal to the axis 68 along which the ram 74 moves. This establishes a coupled engagement between the ram coupler 76 and the syringe plunger coupler 96 in the above-noted manner.

The power injectors 10, 40 of FIGS. 1 and 2A-C each may be used for any appropriate application, including without limitation for medical imaging applications where fluid is injected into a subject (e.g., a patient) and/or any appropriate medical diagnostic and/or therapeutic application (e.g., injection of chemotherapy, pain management, etc.). Representative medical imaging applications for the power injectors 10, 40 include without limitation computed tomography or CT imaging, magnetic resonance imaging or MRI, single photon emission computed tomography or SPECT imaging, positron emission tomography or PET imaging, X-ray imaging, angiographic imaging, optical imaging, and ultrasound imaging. The power injectors 10, 40 each could be used alone or in combination with one or more other components. The power injectors 10, 40 each may be operatively interconnected with one or more components, for instance so that information may be conveyed between the power injector 10, 40 and one or more other components (e.g., scan delay information, injection start signal, injection rate).

Any number of syringes may be utilized by each of the power injectors 10, 40, including without limitation single-head configurations (for a single syringe) and dual-head configurations (for two syringes). In the case of a multiple syringe configuration, each power injector 10, 40 may discharge fluid from the various syringes in any appropriate manner and according to any timing sequence (e.g., sequential discharges from two or more syringes, simultaneous discharges from two or more syringes, or any combination thereof). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). Each such syringe utilized by each of the power injectors 10, 40 may include any appropriate fluid (e.g., a medical fluid), for instance contrast media, therapeutic fluid, a radiopharmaceutical, saline, and any combination thereof. Each such syringe utilized by each of the power injectors 10, 40 may be installed in any appropriate manner (e.g., rear-loading configurations may be utilized; front-loading configurations may be utilized; side-loading configurations may be utilized).

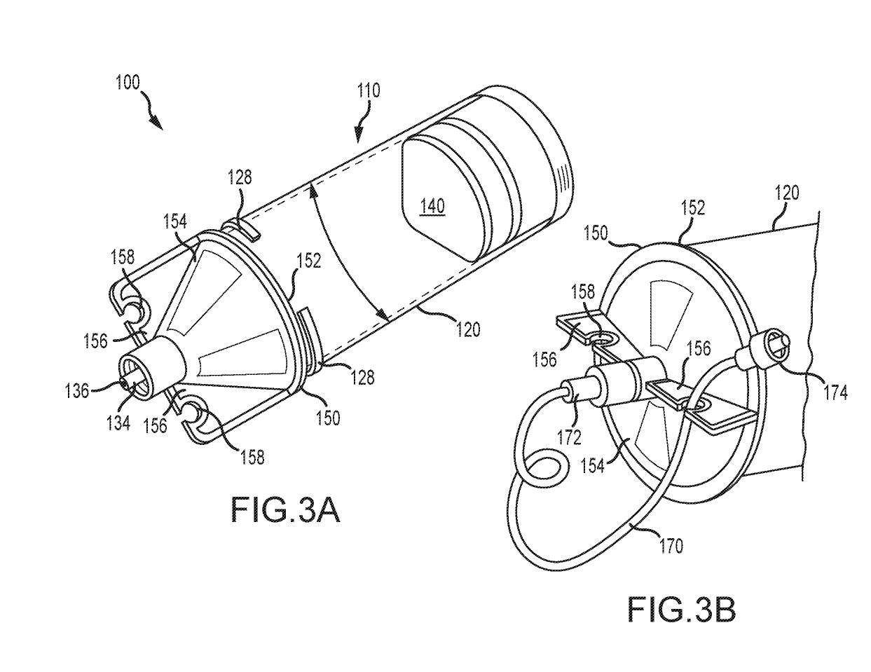

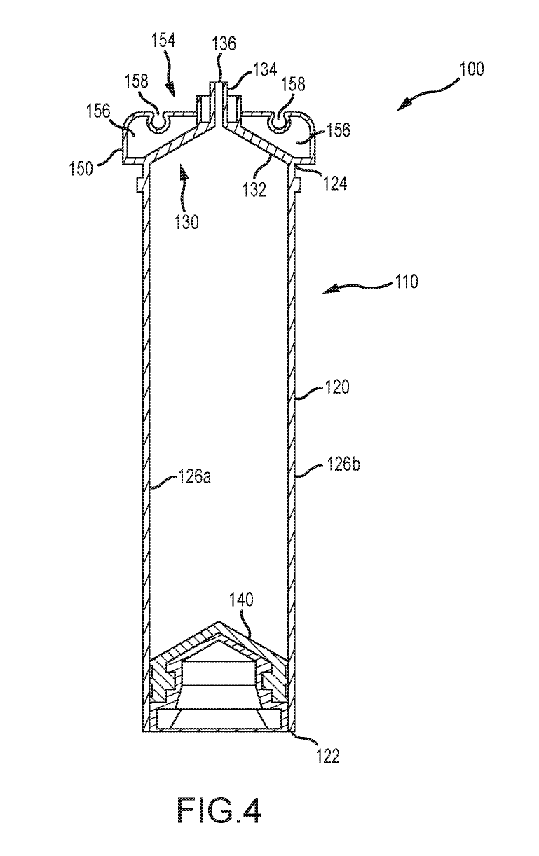

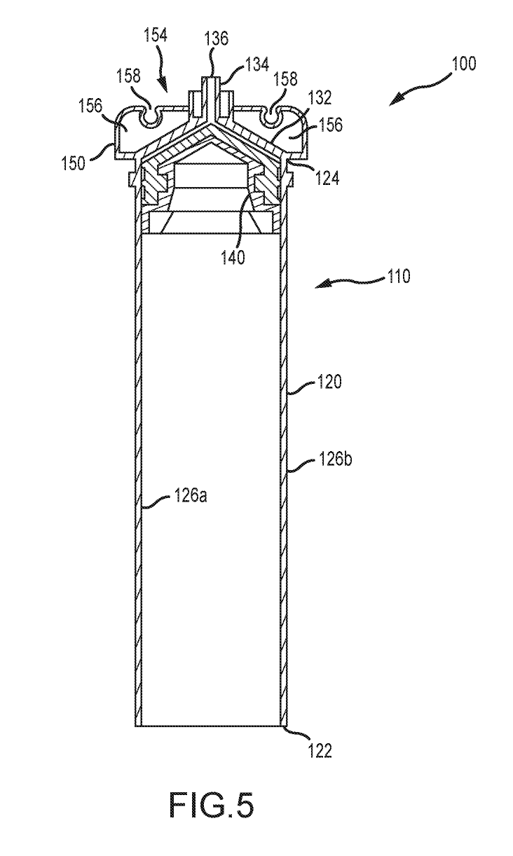

One embodiment of a power injector syringe assembly is addressed in FIGS. 3A-7 and is identified by reference numeral 100. Principal components of the power injector syringe assembly 100 include a syringe body 110, a piston 140 that is movably disposed within the syringe body 110, and a pressure sleeve 180 in which at least a portion of the syringe body 110 is disposed. The power injector syringe assembly 100 may be detachably installed on any appropriate power injector and in any appropriate manner, for instance in accordance with U.S. Pat. No. 7,998,133, the entire disclosure of which is incorporated by reference.

Referring primarily to FIGS. 3A, 3B, 4 and 5, the syringe body 110 includes a syringe barrel 120 and what may be characterized as a discharge end section 130 that extends from the syringe barrel 120 in the illustrated embodiment. Both the syringe barrel 120 and the discharge end section 130 may be characterized as being disposed about a central axis 104 for the syringe body 110. A length dimension for the syringe body 110, and thereby a length dimension for each of the syringe barrel 120 and the discharge end section 130, coincides with (and may be measured along) the central axis 104.

The syringe barrel 120 includes a first end 122 and an oppositely disposed second end 124, the spacing between which defines the length of the syringe barrel 120. The first end 122 may be characterized as a proximal end of the syringe barrel 120, and will be disposed at least generally at the powerhead for the power injector in the installed position. The second end 124 may be characterized as a distal end of the syringe barrel 120, and will be spaced further from the powerhead of the power injector in the installed position (compared to the first end 122 of the syringe barrel 20). The syringe barrel 120 has a plurality of outwardly projecting mounting flange/lug sections 128 that are spaced (e.g., equally) around the circumference of the syringe barrel 120 near or at its second or distal end 124. These mounting flange sections 128 may be equally spaced about the circumference of the syringe barrel 120, but may be disposed in any appropriate arrangement that allows these mounting flange sections 128 to be used to secure the syringe body 110 to the pressure sleeve 180 in a manner that will be discussed in more detail below.

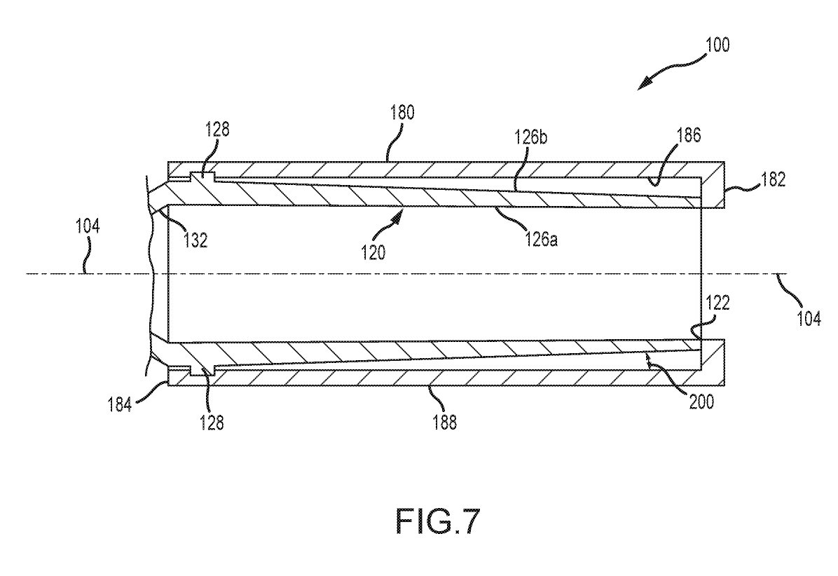

An inner wall 126a for the syringe barrel 120 extends from its first end 122 to its second end 124. This inner wall 126a is cylindrical, and thereby is of a constant diameter proceeding from the first end 122 of the syringe barrel 122 to its second end 124. An outer wall 126b for the syringe barrel 120 also extends from its first end 122 to its second end 124. At least part of this outer wall 126b for the syringe barrel 120 is tapered in diverging relation relative to the central axis 104 in proceeding in a direction of the second end 122 of the syringe barrel 120. In the illustrated embodiment, the entirety of the outer wall 126b for the syringe barrel 120 is tapered in diverging relation relative to the central axis 104 in proceeding from the first end 122 of the syringe barrel 120 to the second end 124 of the syringe barrel 120, and this taper is of a constant or fixed magnitude. FIG. 7 shows a taper angle 200 for the outer wall 126b of the syringe barrel 120. One embodiment has the taper angle 200 being of a constant magnitude of about 5°. In another embodiment, the taper angle 200 does not exceed 10°. Further, in another embodiment, this taper angle 200 is a constant magnitude and is within a range of about 0.5° to about 10°.

The above-noted taper of the outer wall 126b of the syringe barrel 120 is subject to a number of characterizations. One is that the outer diameter of the syringe barrel 120 continually increases proceeding from a first location (e.g., coinciding with the first end 122 for the syringe barrel 120) to a second location (e.g., coinciding with the second end 124 for the syringe barrel 120). Another is that the wall thickness of the syringe barrel 120 continually increases proceeding from a first location (e.g., coinciding with the first end 122 for the syringe barrel 120) to a second location (e.g., coinciding with the second end 124 for the syringe barrel 120).

The discharge end section 130 for the syringe body 110 extends from the second end 124 of the syringe barrel 120 (in a direction that is also away from the first end 122 of the syringe barrel 120). This discharge end section 130 includes a transition section 132 and a discharge nozzle 134. The transition section 132 proceeds from the second end 122 of the syringe barrel 120 in converging relation relative to the central axis 104, and may be characterized as frustumly-shaped (e.g., in the form of a truncated cone). The discharge nozzle 134 extends from this transition section 132 and incorporates a discharge orifice 136 on its free end. Movement of the piston 140, within the syringe barrel 120 and in the direction of the second end 124 of the syringe barrel 120, will discharge fluid out of the syringe body 110 through this discharge orifice 136 (where this fluid is initially located between the piston 140 and the discharge orifice 136).

The syringe body 110 may also incorporate a shield or cap 150, and the shield 150 is integrally formed with the syringe body 110 in the illustrated embodiment. The shield 150 may be characterized as being part of the discharge end section 130 of the syringe body 110. It should be appreciated that the shield 150 could also be separately formed from the syringe body 120 and still be part of the power injector syringe assembly 100, and may be secured relative to the syringe body 110 in any appropriate manner (including via a detachable connection). For instance, the shield 150 could be integrated in the manner of the cap 51 that is addressed in the above-noted U.S. Pat. No. 7,998,133.

The shield 150 has an outer diameter that is larger the outer diameter of the pressure sleeve 180 (again, that may be used by the power injector syringe assembly 100 and that is discussed in more detail below). This larger outer diameter for the shield 150 should reduce the potential for fluids being able to enter the open space between the inner diameter of the pressure sleeve 180 and the outer diameter of the syringe barrel 120. In the illustrated embodiment, the shield 150 also incorporates at least one tubing clip 156. Two tubing clips 156 are shown for the illustrated embodiment. Any appropriate number of one or more tubing clips 156 may be utilized, and multiple tubing clips 156 may be disposed in any appropriate relative arrangement. In any case, each tubing clip 156 includes a tube receptacle 158 that is both concave and accessible on an exterior of the power injector syringe assembly 100 when in an installed configuration.

At least one of the noted tubing clips 156 is subject to a number of characterizations. The shield 150 may be characterized as having a first side 152 (a first cap side) that faces or projects in the direction of the first end 122 of the syringe barrel 120, along with a second side 154 (a second cap side) that faces or projects in the opposite direction compared to the first side 152. One or more tubing clips 156 may be incorporated by the second side 154 of the shield 150 (e.g., on an exterior of the power injector syringe assembly 100). At least one tubing clip 156 may be characterized as being incorporated such that is corresponding tube receptacle 158 is disposed beyond the transition section 132 and such that the second end 124 of the syringe barrel 120 is located between this tube receptacle 158 and the first end 122 of the syringe barrel 120 (“between” being relative to the central axis 104). At least one tubing clip 156 may be characterized as being disposed beyond or outside of the pressure sleeve 180 for an installed configuration of the power injector syringe assembly 100 and as will be addressed in more detail below.

One or more tubing clips 156 could be incorporated by the power injector syringe assembly 100 even if the above-described shield 150 is not used by the power injector syringe assembly 100. For instance, one or more tubing clips 156 could extend from an exterior of the transition section 132 of the syringe body 110 (e.g., proceeding in a direction that is at least generally away from the syringe barrel 120). It would then be proper to consider each such tubing clip 156 as being part of the discharge end section 130 of the syringe body 110.

FIG. 3B illustrates that the power injector syringe assembly 100 may further include an extension tube 170 having a first tube end 172 and a second tube end 174. The first tube end 172 may be secured to the discharge nozzle 134 of the syringe body 110 (e.g., utilizing a Luer connector or the like). The second tube end 174 is spaced from the first tube end 172, and may include an appropriate connector (e.g., a Luer connector) for detachable connection with a tubing set (not shown, but that would extend to a patient). A portion of the extension tube 170, located between the first tube end 172 and the second tube end 174, is detachably received in the tube receptacle 158 of one of the tubing clips 156 in the illustrated embodiment. This accommodates having the extension tube 170 being installed on the syringe body 110 prior to fluidly connecting the extension tube 170 with a tubing set (e.g. a tubing clip 156 provides a stowed position for the extension tube 170 while installed on the syringe body 110, when the power injector syringe assembly 100 is installed on a power injector, and prior to fluidly connecting the extension tube with a patient).

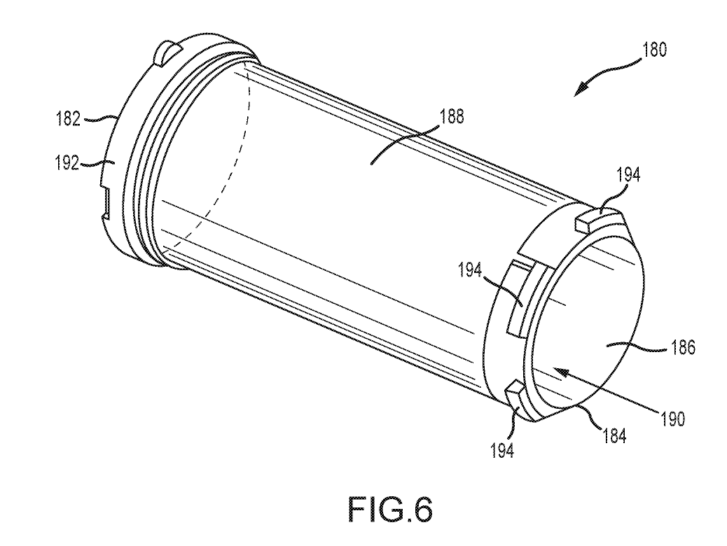

The illustrated syringe body 110 is configured for use with a pressure sleeve or jacket in the illustrated embodiment. A representative pressure sleeve that may be incorporated by the power injector syringe assembly 100 is illustrated in FIG. 6 and is identified by reference numeral 180. Generally, the pressure sleeve 180 may be configured to provide a higher pressure resistance than the syringe body 110. As such, the pressure sleeve 180 may be repeatedly used—the pressure sleeve 180 may be used for multiple injections, including where each syringe body 110 is only used for a single injection.

The syringe body 110 may be configured to withstand only moderate or low pressure via power injector operations, and thereby may be referred to as a disposable (e.g., usable for only a single injection procedure). The walls of the syringe body 110 are usually outwardly deformable under power injector operating pressures, particularly pressures of 300 psi or more. Such higher pressures are necessary to overcome pressure drops through the injection tubing at higher flow rates, which are often desirable for the execution of various types of injection protocols by the power injector. The pressure sleeve 180 may be made of a stronger material that will withstand these operating pressures. When the syringe body 110 is contained in the pressure sleeve 180, it's syringe barrel 120 is surrounded by the pressure sleeve 180 and is supported by the pressure sleeve 180 against expansion caused by the fluid pressure within as the syringe barrel 110 expands against the pressure sleeve 180.

The pressure sleeve 180 includes a first end 182 (e.g., a proximal end), and an oppositely disposed second end 184 (e.g., a distal end 184). An inner wall 186 and outer wall 190 for the pressure sleeve 180 are each of a cylindrical configuration and are concentrically disposed about the central axis 104. As such, the pressure sleeve 180 has an at least substantially constant wall thickness proceeding from its first end 182 to its second end 184. This wall thickness of the pressure sleeve 180 (and/or the material used for the pressure sleeve 180) structurally reinforces the syringe barrel 120 during power injector operations, particularly at higher operating pressures.

The pressure sleeve 180 has a cylindrical inner bore 190 extending therethrough from its first or proximate end 182 to second or distal end 184. The inner bore 190 is dimensioned so as to receive the syringe body 110 (more specifically the syringe barrel 120) through the second end 184 of the pressure sleeve 180 (i.e., the syringe body 110 is “front loaded” into the pressure sleeve 180) and to support the syringe barrel 120 against “over expansion” from fluid pressure within the syringe body 110. The pressure sleeve 180 has an annular flange 192 extending outwardly around its first or proximate end 182. The flange 192 may be integrally formed with the jacket cylinder and is shaped for installation on a powerhead of a power injector (e.g., in accordance with U.S. Pat. No. 7,998,133, noted above).

The pressure sleeve 180 has a plurality of outwardly projecting mounting flange/lug sections 194 (four in the illustrated embodiment, although any appropriate number may be utilized) that are spaced around the circumference of the pressure sleeve 180 near its second or remote end 184. These mounting flange sections 194 may be equally spaced about the circumference of the pressure sleeve 180. In any case, the syringe body 110 may be inserted into the pressure sleeve 180 (the first end 122 of the syringe barrel 120 being directed through the second end 184 of the pressure sleeve 180) such that each of its mounting flange sections 128 is disposed between an adjacent pair of mounting flange sections 194 for the pressure sleeve 180. After the syringe body 110 has been fully inserted into the pressure sleeve 180, the syringe body 110 may then be rotated relative to the pressure sleeve 180 to lock the syringe body 110 to the pressure sleeve 180 (e.g., to dispose each mounting flange section 128 of the syringe barrel 120 at least partially “behind” a corresponding mounting flange section 194 of the pressure sleeve 180). At this time, the second end 184 of the pressure sleeve 180 may be disposed against (or in closely spaced relation to) the first side 152 of the cap 150 (and in any case the noted shield 150 will extend radially beyond the outer wall 188 of the pressure sleeve 180, as addressed above). As noted above and at this time, at least one tubing clip 156 will also be disposed beyond the second or distal end 184 of the pressure sleeve 180 in a direction that is away from the syringe barrel 120. Stated another way, at least one tubing clip 156 for the power injector syringe assembly 100 will be disposed outside of the pressure sleeve 180 and accessible on an exterior for receipt of a tube being utilized by the power injector syringe assembly 100 (e.g., extension tube 170).

Based upon the configuration/orientation of the outer wall 126b of the syringe barrel 120 (e.g., tapered), the spacing between the outer wall 126b of the syringe barrel 120 and the inner wall 186 of the pressure sleeve 180 changes when proceeding from the first end 122 of the syringe barrel 120 to the second end 124 of the syringe barrel 120, including where this spacing changes at a constant or uniform rate and as schematically shown in FIG. 7. The smallest spacing between the outer wall 126b of the syringe barrel 120 and the inner wall 186 of the pressure sleeve 180 is thereby at the discharge end of the syringe barrel 120 (coinciding with its second end 124). The outer wall 126b of the syringe barrel 120 could actually be in contact with the outer wall 188 of the pressure sleeve 180 at the second end 124 of the syringe barrel 120 (not shown).

The noted clearance between the syringe barrel 120 and the pressure sleeve 180 provides a number of advantages. Initially, the maximum clearance between the syringe barrel 120 and the pressure jacket 180 is encountered when initially introducing the first end 122 of the syringe barrel 120 into the second end 184 of the pressure sleeve 180. This should facilitate the loading of the syringe barrel 120 into the pressure sleeve 180 (again, where this pressure sleeve 180 will typically already have been installed on the powerhead of a power injector). Conversely, the minimum clearance between the syringe barrel 120 and the pressure jacket 180 will be at the second end 124 of the syringe barrel 120, and which may experience higher fluid pressures during power injector operations.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A power injector syringe assembly comprising:
a syringe body comprising:
a syringe barrel comprising an inner diameter and an outer diameter that are each disposed about a central axis of said syringe body, wherein said outer diameter continually increases proceeding from a first location to a second location; and
a discharge orifice; and
a piston movably disposed within said syringe barrel, wherein said second location and said discharge orifice are spaced from said first location in a common direction.

2. The power injector syringe assembly of claim 1, wherein said syringe barrel comprises an outer wall and an inner wall, wherein said inner wall is cylindrical, wherein said outer wall comprises a taper that proceeds from said first location to said second location, and wherein said taper is measured relative to an axis that is parallel to said central axis of said syringe body.

3. The power injector syringe assembly of claim 2, wherein said taper exists along an entire length of said syringe barrel.

4. The power injector syringe assembly of any of claim 3, wherein said taper is of a constant, fixed magnitude.

5-8. (canceled)

9. The power injector syringe assembly of claim 1, wherein said first location is an open end of said syringe body that is opposite of a discharge end of syringe body that comprises said discharge orifice.

10. (canceled)

11. The power injector syringe assembly of claim 1, wherein said syringe barrel comprises first and second ends that are opposite of each other and that are spaced along said central axis, wherein said first end of said syringe barrel defines a proximal end of said syringe body, and wherein syringe body further comprises a frustumly-shaped section that extends from said second end of said syringe barrel and that converges toward said central axis of said syringe body in proceeding away from said syringe barrel, wherein said first end of said syringe barrel comprises said first location, and wherein said second end of said syringe barrel comprises said second location.

12. (canceled)

13. The power injector syringe assembly of claim 11, wherein said outer diameter of said syringe barrel continuously diverges relative to said central axis in proceeding from said first end of said syringe barrel to said second end of said syringe barrel.

14. The power injector syringe assembly of claim 13, wherein said inner diameter of said syringe barrel is of a constant magnitude from said first end of said syringe barrel to said second end of said syringe barrel.

15-16. (canceled)

17. The power injector syringe assembly of claim 11, further comprising:
a shield disposed over an exterior of said frustumly-shaped section; and
a first tubing clip disposed on an exterior of said shield, wherein said first tubing clip comprises a first tube receptacle.

18. (canceled)

19. The power injector syringe assembly of claim 17, wherein said first tube receptacle comprises a concave opening on an exterior of said power injector syringe assembly.

20. (canceled)

21. The power injector syringe assembly of claim 17, further comprising an extension tube that extends from said syringe body and that is detachably received within said first tube receptacle, wherein a first tubing segment of said extension tube is positioned between said syringe body and said first tubing clip, and wherein a second tubing segment of said extension tube proceeds beyond said first tubing clip and comprises a free end for said extension tube.

22-26. (canceled)

27. The power injector syringe assembly of claim 1, wherein said syringe barrel comprises a plurality of locking lugs that are spaced about said central axis of said syringe body and that are on a said discharge end portion of said syringe barrel.

28. The power injector syringe assembly of claim 1, further comprising a first tubing clip.

29. The power injector syringe assembly of claim 28, wherein said first tube receptacle comprises a concave opening on an exterior of said power injector syringe assembly.

30. (canceled)

31. The power injector syringe assembly of claim 28, further comprising an extension tube that extends from said syringe body and that is detachably received within said first tube receptacle, wherein a first tubing segment of said extension tube is positioned between said syringe body and said first tubing clip, and wherein a second tubing segment of said extension tube proceeds beyond said first tubing clip and comprises a free end for said extension tube.

32. The power injector syringe assembly of claim 1, further comprising:
a pressure sleeve, wherein said syringe barrel is disposed within said pressure sleeve.

33. The power injector syringe assembly of claim 32, wherein said pressure sleeve comprises an inner diameter and an outer diameter, wherein said inner diameter of said pressure sleeve is of a constant magnitude for a location corresponding with said first location of said syringe barrel to a location corresponding with said second location of said syringe barrel.

34. The power injector syringe assembly of claim 32, wherein a spacing between said syringe barrel and said pressure sleeve continually decreases proceeding from said first location of said syringe barrel to said second location of said syringe barrel.

35. The power injector syringe assembly of claim 32, wherein a spacing between said syringe barrel and said pressure sleeve decreases at a constant rate proceeding from said first location of said syringe barrel to said second location of said syringe barrel.

36. The power injector syringe assembly of claim 32, further comprising a first tubing clip that is disposed beyond and outside of said pressure sleeve.

37. The power injector syringe assembly of claim 36, wherein said first tube receptacle comprises a concave opening on an exterior of said power injector syringe assembly.

38. (canceled)

39. The power injector syringe assembly of claim 37, further comprising an extension tube that extends from said syringe body and that is detachably received within said first tube receptacle, wherein a first tubing segment of said extension tube is positioned between said syringe body and said first tubing clip, and wherein a second tubing segment of said extension tube proceeds beyond said first tubing clip and comprises a free end for said extension tube.
